(19) United States
(12) Patent Application Publication
(10) Pub. No.: US 2002/0025926 A1
(43) Pub. Date: Feb. 28, 2002

(54) Enantiomers of S-adenosyl-l-methionine

(76) Inventor: Rolland F. Hebert, Seattle, WA (US)

Correspondence Address: ROLLAND HEBERT, 427 BELLEVUE AVE E. SUITE 301, SEATTLE, WA, 98102, US

(21) Appl. No.: 09/943,243

(22) Filed: Aug. 30, 2001

Related U.S. Application Data
(60) Provisional application No. 60/229,151, filed on Aug. 30, 2000.

(51) Int. Cl. A61K 38/13; A61K 31/7076; A61K 31/525

(52) U.S. Cl. 514/9; 514/46; 514/251; 514/161

(57) ABSTRACT

Enantiomers of S-adenosyl-l-methionine, their stable salts and their uses are described. These compositions possess potent activity in treating various conditions involving hypomethylation and transulfuration reactions and are valuable for use as active constituents in pharmaceutical compositions.

### BACKGROUND-CROSS-REFERENCES TO RELATED APPLICATION

This application claims the benefit of Provisional Patent Application Ser. No.: 60/229151 filed on Aug. 30, 2000.

### FIELD OF THE INVENTION

The present invention relates to novel compositions of matter containing optically pure enantiomers of S-adenosyl-l-methionine. These compositions possess potent activity in treating various conditions involving hypomethylation and transulfuration reactions.

### TECHNICAL FIELD

This patent relates to novel compositions of matter containing optically pure enantiomers of S-adenosyl-l-methionine (SAM-e) and to therapeutic uses of these new compositions. More particularly, the invention relates to the substantially optically pure enantiomer (S,S)-S-adenosylmethionine, pharmaceutically acceptable salts and pharmaceutical compositions that contain them as active principles.

### BACKGROUND OF THE INVENTION

Many organic compounds exist in optically active forms, i.e., they have the ability to rotate the plane of plane-polarized light. In describing an optically active compound, the prefixes D and L or R and S are used to denote the absolute configuration of the molecule about its chiral center.

The prefixes d and l or (+) and (−) are employed to designate the sign of rotation of planepolarized light by the compound, with (−) or l meaning that the compound is levorotatory. A compound prefixed with (+) or d is dextrorotatory. For a given chemical structure, these compounds, called stereoisomers, are identical except that they are mirror images of one another. A specific stereoisomer may also be referred to as an enantiomer, and a mixture of such isomers is often called an enantiomeric mixture. A 50:50 mixture of enantiomers is referred to as a racemic mixture.

Stereochemical purity is of importance in the field of pharmaceuticals, where 12 of the 20 most prescribed drugs exhibit chirality. A case in point is provided by the L-form of the betaadrenergic blocking agent, propranolol, which is known to be 100 times more potent than the D-enantiomer.

Furthermore, optical purity is important since certain isomers may actually be deleterious rather than simply inert. For example, it has been suggested that the D-enantiomer of thalidomide was a safe and effective sedative when prescribed for the control of morning sickness during pregnancy, and that the corresponding L-enantiomer was a potent teratogen.

S-adenosyl-l-methionine is a naturally occurring substance that is present in all living organisms and has a number of very important biological functions. Among these functions are the following: methyl group donor in transmethylation reactions (it is the sole methyl group donor in such reactions-including methylation of DNA, proteins, hormones, catechol and indoleamines and phosphatidylethanolamine to phosphatidylcholine); it is a substrate of an enzyme lyase that converts S-adenosyl-l-methionine to the molecule methylthioadenosine and homoserine; it is an aminobutyric chain donor to tRNA; it is an aminoacidic chain donor in the biosynthesis of biotin; SAM-e, after decarboxylation, is the donor of aminopropyl groups for the biosynthesis of neuroregulatory polyamines spermidine and spermine. (Zappia et al (1979), Biomedical and Pharmacologcial roles of Adenosylmethionine and the Central Nervous System, page 1, Pergamon Press. N.Y.)

SAM-e has been used clinically in the treatment of liver disease (Friedel H, Goa, K. L., and Benfield P., (1989), S-Adenosyl-l-methionine: a review of its pharmacological properties and therapeutic potential in liver dysfunction and affective disorders in relation to its physiological role in cell metabolism. Drugs. 38, 389-416), arthritis (Di Padova C, (1987), S-adenosyl-l-methionine in the treatment of osteoarthritis: review of the clinical studies. Am J. Med. 83, (Suppl. 5), 6-65), and depression (Kagan, B, Sultzer D. L., Rosenlicht N and Gerner R. (1990), Oral S-adenosylmethionine in depression: a randomized, double blind, placebo-controlled trial. Am. J. Psychiatry 147, 591-595.) Alzheimer's patients have reduced cerebral spinal fluid levels of S-adenosyl-l-methionine (Bottiglieri et al, (1990), Cerebrospinal fluid S-adenosyl-l-methionine in depression and dementia: effects of treatment with parenteral and oral S-adenosyl-l-methionine. J. Neurol. Neurosurg. Psychiatry 53, 1096-1098.) In a preliminary study, SAM-e was able to produce cognitive improvement in patients with Alzheimer's disease. (Bottiglieri et al (1994), The clinical potential of admetionine (S-adenosyl-l-methioinine) in neurological disorders. Drugs 48, 137-152.) SAM-e brain levels in patients with Alzheimer's disease are also severely decreased. (Morrison et al, (1996), Brain S-adenosylmethionine levels are severely decreased in Alzheimer's disease, Journal of Neurochemistry, 67, 1328-1331.) Patients with Parkinson's disease have also been shown to have significantly decreased blood levels of SAM-e. (Cheng et al, (1997), Levels of L-methionine S-adenosyltransferase activity in erythrocytes and concentrations of S-adenosylmethionine and S-adenosylhomocysteine in whole blood of patients with Parkinson's disease. Experimental Neurology 145, 580-585.)

SAM-e levels in patients treated with the antineoplastic drug methotrexate are reduced. Neurotoxicity associated with this drug may be attenuated by co-administration of SAM-e. (Bottiglieri et al (1994), The Clinical Potential of Ademetionine (S-adenosylmethionine) in neurological disorders, Drugs, 48 (2), 137-152.)

Cerebral spinal fluid levels of SAM-e have been investigated in HIV AIDS dementia Complex/HIV encephalopathy and found to be significantly lower than in non-HIV infected patients. (Keating et al (1991), Evidence of brain methyltransferase inhibition and early brain involvement in HIV positive patients Lancet: 337:935-9.)

De La Cruz et al have shown that SAM-e, chronically administered, can modify the oxidative status in the brain by enhancing anti-oxidative defenses. (De La Cruz et al, (2000), Effects of chronic administration of S-adenosyl-l-methionine on brain oxidative stress in rats. Naunyn-Schmiedeberg's Archives Pharmacol 361: 47-52.) This is similar to results obtained with SAM-e in liver and kidney tissue. Thus SAM-e would be useful as an antioxidant.

Oral SAM-e administration to patients with and without liver disease has resulted in increases in liver glutathione levels. (Vendemiale G et al, (1989), Effect of oral S-adenosyl-l-methionine on hepatic glutathione in patients with liver disease. Scand J Gastroenterol; 24: 407-15. Oral administration of SAM-e to patients suffering from intrahepatic cholestasis had improvements in both the pruritus as well as the biochemical markers of cholestasis. (Giudici et al, The use of admethionine (SAM-e) in the treatment of cholestatic liver disorders. Meta-analysis of clinical trials. In: Mato et al editors. Methionine Metabolism: Molecular Mechanism and Clinical Implications. Madrid: CSIC Press; 1992 pp 67-79.) Oral SAM-e administration to patients suffering from primary fibromyalgia resulted in significant improvement after a short term trial. (Tavoni et al, Evaluation of S-adenosylmethioine in Primary Fibromaylgia. The American Journal of Medicine, Vol 83 (suppl 5A), pp 107-110, 1987.) SAM-e has been used for the treatment of osteoarthritis as well. (Koenig B. A long-term (two years) clinical trial with S-adenosylmethionine for the treatment of osteoarthritis. The American Journal of Medicine, Vol 83 (suppl 5A), Nov. 20, 1987 pp 89-94)

SAM-e is clinically useful in many apparently unrelated areas because of its important function in basic metabolic processes. One of its most striking clinical uses is in the treatment of alcoholic liver cirrhosis that, until now, remained medically untreatable. Mato et al demonstrated the ability of oral SAM-e in alcoholic liver cirrhosis to decrease the overall mortality and/or progression to liver transplant by 29% vs 12% as compared with a placebo treated group. (Mato et al (1999), S-adenosylmethionine in alcohol liver cirrhosis: a randomized, placebo-controlled, double blind, multi-center clinical trial, Journal of Hepatology, 30, 1081-1089.)

Sam-e also attenuates the damage caused by tumor necrosis factor alpha and can also decrease the amount of tumor necrosis factor alpha secreted by cells. Consequently, conditions in which this particular inflammatory factor is elevated would benefit from the administration of SAM-e. (Watson WH, Zhao Y, Chawla RK, (1999) Biochem J Aug. 15; 342 (Pt 1):21-5. S-adenosylmethionine attenuates the lipopolysaccharide-induced expression of the gene for tumour necrosis factor alpha.) SAM-e has also been studied for its ability to reduce the toxicity associated with administration of cyclosporine A, a powerful immunosuppressor. (Galan A, et al, Cyclosporine A toxicity and effect of the s-adenosylmethionine, Ars Pharmaceutica, 40:3; 151-163, 1999.)

SAM-e, incubated in vitro with human erythrocytes, penetrates the cell membrane and increases ATP within the cell thus restoring the cell shape. (Friedel et al, S-adenosyl-l-methionine: A review of its pharmacological properties and therapeutic potential in liver dysfunction and affective disorders in relation to its physiological role in cell metabolism, Drugs 38 (3):389-416, 1989)

SAM-e has been studied in patients suffering from migraines and found to be of benefit. (Friedel et al, S-adenosyl-l-methionine: A review of its pharmacological properties and therapeutic potential in liver dysfunction and affective disorders in relation to its physiological role in cell metabolism, Drugs 38 (3): 389-416, 1989)

SAM-e has been administered to patients with peripheral occlusive arterial disease and was shown to reduce blood viscosity, chiefly via its effect on erythrocyte deformability.

SAM-e is commercially available using fermentation technologies that result in SAM-e formulations varying between 60 and 80% purity. (That is, the final product contains 60-80% of the active or (S, S)-SAM-e and 20-40% of the inactive or (R, S) -SAM-e.) (Gross, A., Geresh, S., and Whitesides, Gm (1983) Appl. Biochem. Biotech. 8, 415.) Enzymatic synthetic methodologies have been reported to yield the inactive isomer in concentrations exceeding 60%. (Matos, JR, Rauschel FM, Wong, CH. S-Adenosylmethionine: Studies on Chemical and Enzymatic Synthesis. Biotechnology and Applied Biochemistry 9, 39-52 (1987). Enantiomeric separation technologies have been reported to resolve the pure active enantiomer of SAM-e. (Matos, JR, Rauschel FM, Wong, CH. S-Adenosylmethionine: Studies on Chemical and Enzymatic Synthesis. Biotechnology and Applied Biochemistry 9, 39-52 (1987; Hoffman, Chromatographic Analysis of the Chiral and Covalent Instability of S-adenosyl-l-methionine, Biochemistry 1986, 25 4444-4449: Segal D and Eichler D, The Specificity of Interaction between S-adenosyl-l-methionine and a nucleolar 2-0-methyltransferase, Archives of Biochemistry and Biophysics, Vol. 275, No. 2, December, pp. 334-343, 1989) Newer separation technologies exist to resolve enantiomers on a large commercial production scale at a very economic cost. In addition, it would be conceivable to synthesize the biologically active enantiomer using special sterioselective methodologies but this has not been accomplished to date.

De la Haba first showed that the sulfur is chiral and that only one of the two possible configurations was synthesized and used biologically. (De la Haba et al J. Am. Chem. Soc. 81, 3975-3980, 1959) Methylation of RNA and DNA is essential for normal cellular growth. This methylation is carried out using SAM-e as the sole or major methyl donor with the reaction being carried out by a methyltransferase enzyme. Segal and Eichler showed that the enzyme bound (S, S)-SAM-e 10 fold more tightly than the biologically inactive (R, S)-SAM-e thus demonstrating a novel binding stereospecificity at the sulfur chiral center. Other methyltransferases have been reported to bind (R, S)-SAM-e to the same extent as (S, S)-SAM-e and thus (R, S)-SAM-e could act as a competitive inhibitor of that enzyme. (Segal D and Eichler D, The Specificity of Interaction between S-adenosyl-l-methionine and a nucleolar 2-0-methyltransferase, Archives of Biochemistry and Biophysics, Vol. 275, No. 2, December pp. 334-343, 1989; Borchardt RT and Wu YS , Potential inhibitors of S-adenosylmethionine-dependent methyltransferases. Role of the Asymmetric Sulfonium Pole in the Enzymatic binding of S-adenosyl-l-methionine, Journal of Medicinal Chemistry, 1976, Vol 19, No. 9, 1099-1103.)

SAM-e (whether in its optically pure enantiomeric form or in an enantiomeric or racemic mixture) presents certain difficult problems in terms of its stability at ambient temperature that result in degradation of the molecule to undesirable degradation products. SAM-e (and thus its enantiomers) must be further stabilized since it exhibits intramolecular instability that causes the destabilization and breakdown of the molecule at both high as well as ambient temperatures. SAM-e has therefore been the subject of many patents directed both towards obtaining new stable salts, and towards the provision of preparation processes that can be implemented on an industrial scale. The present patent thus envisions the use of any of the salts of SAM-e already disclosed in the prior art to stabilize the enantiomeric forms of SAM-e.

### PRIOR ART

Many patents exist disclosing salts of SAM-e that stabilize the molecule but none discloses the use of an optically pure enantiomer of SAM-e. U.S. Pat. No. 2,969,353, Shunk et al, Jan. 24, 1962, discloses a method for the preparation of Sam-e and a stable salt of SAM-e but not the use of an optically pure enantiomer of SAM-e. U.S. Pat. No. 3,707,536, Haid et al, Dec. 26, 1972, discloses a new SAM-e bisulfate salt but not the use of an optically pure enantiomer of SAM-e. U.S. Pat. No. 3,893,999, Fiecchi, Jul. 8, 1975, discloses a new salt of SAM-e made with tri-p-toluensulphonate but not the use of an optically pure enantiomer of SAM-e. U.S. Pat. No. 5,102,791, Gennari, Apr. 7, 1992, discloses, among others, a 1,4 butanedisulfonate salt of SAM-e but not the use of an optically pure enantiomer of SAM-e. U.S. Pat. No. 4,028,183, Fiecchi, Jun. 7, 1977, discloses, among others, p-toluene sulfonate as a means to stabilize the SAM-e molecule but does not disclose the use of an optically pure enantiomer of SAM-e. U.S. Pat. No.4,764,603, Zappia, Aug. 16, 1988, discloses the use of polyanions such as polyphosphates, polyvinylsulfonates-sulfates or phosphates, polyacrylates, and polystyrene sulfonates. However, this patent does not disclose the use of an optically pure enantiomer of SAM-e.

U.S. Pat. No. 6,117,849, Zimmermann, et al. Sep. 12, 2000, discloses the use of SAM-e complexed with nucleosides as HIV inhibitors but does not disclose the use of an optically pure enantiomer of SAM-e for any other condition nor the enantiomer of SAM-e uncomplexed to another molecule. U.S. Pat. No. 4,465,672, Gennari, Aug. 14, 1984, discloses new SAM-e salts but does not disclose the use of an optically pure enantiomer of SAM-e. U.S. Pat. No. 3,954,726, Fiecchi, May 4, 1976, discloses double salts of SAM-e but does not disclose the use of an optically pure enantiomer of SAM-e. U.S. Pat. No. 4,057,686, Fiecchi, Nov. 8, 1977, discloses stable salts of SAM-e but does not disclose the use of an optically pure enantiomer of SAM-e. U.S. Pat. No. 4,109,079 Kawahara, et al., Aug. 22, 1978, discloses new stable SAM-e salts but does not disclose the use of an optically pure enantiomer of SAM-e. U.S. Pat. No. 4,242,505, Kawahara, et al. Dec. 30, 1980, discloses new stabilizing salts of SAM-e but does not disclose the use of an optically pure enantiomer of SAM-e. U.S. Pat. No. 4,369,177, Kozaki, et al. Jan. 18, 1983, discloses new stable SAM-e salts but does not disclose the use of an optically pure enantiomer of SAM-e. U.S. Pat. No. 4,543,408, Gennari, Sep. 24, 1985, discloses new SAM-e salts but does not disclose the use of an optically pure enantiomer of SAM-e. U.S. Pat. No. 4,558,122, Gennari, Dec. 10, 1985, discloses new SAM-e salts but does not disclose the use of an optically pure enantiomer of SAM-e. U.S. Pat. No. 4,764,603, Zappia, et al. Aug. 16, 1988, discloses the use of new salts of SAM-e but does not disclose the use of an optically pure enantiomer of SAM-e. U.S. Pat. No. 4,990,606, Gennari, Feb. 5, 1991, discloses new salts of SAM-e but does not disclose the use of an optically pure enantiomer of SAM-e. U.S. Pat. No. 5,073,546, Zappia, et al. Dec. 17, 1991, discloses new salts of SAM-e but does not disclose the use of an optically pure enantiomer of SAM-e. U.S. Pat. No. 5,114,931, Gennari, May 19, 1992, discloses injectable SAM-e salts but does not disclose the use of an optically pure enantiomer of SAM-e. U.S. Pat. No. 5,128,249, Gennari, Jul. 7, 1992, discloses new SAM-e salts but does not disclose the use of an optically pure enantiomer of SAM-e. U.S. Pat. No. 5,196,402, Braganza, et al. Mar. 23, 1993, discloses the use of SAM-e for certain clinical uses but does not disclose the use of an optically pure enantiomer of SAM-e.

U.S. Pat. No. 5,466,678, Kawabata, et al. Nov. 14, 1995, discloses the use SAM-e to decrease the side effects of chemotherapy but does not disclose the use of an optically pure enantiomer of SAM-e to accomplish this. U.S. Pat. No. 5,137,712, Kask et al, Aug. 11, 1992 discloses the use of SAM-e to reverse or prevent side effects of neuroleptic treatment but does not disclose the use of an optically pure enantiomer of SAM-e.

Administration of optically pure enantiomers of SAM-e salts of the present invention would have significant utility over a wide range of disorders or conditions associated with low levels of SAM-e. Since the two enantiomeric forms of S-adenosyl-l-methionine do not exhibit the same biological activity but rather that the (R, S) S-adenosyl-l-methionine enantiomer exhibits no biological activity (or even competitive inhibition), it is therefore necessary for a rational pharmaceutical therapy to use the more active enantiomeric form of S-adenosyl-l methionine. In this regard, and in view of the (R, S)-SAM-e enantiomer to act as a competitive inhibitor of (S, S,) SAM-e in methyltransferase reactions, a more ideal SAM-e composition would be the substantially optically pure biologically active (S, S)-SAM-e form.

It is an object of the present invention to provide new compositions of SAM-e containing substantially pure biologically active (S, S) SAM-e. It is a further object of the present invention to provide such compositions that provide treatment or prevention of conditions that are related to lowering SAM-e levels. It is a still further object of the present invention to provide such compositions having good stability.

Accordingly, there is need in the art for new, substantially optically pure enantiomeric forms of SAM-e as well as methods related to the use of such substantially optically pure enantiomeric forms of SAM-e to increase blood and other tissue and fluid levels of SAM-e and to treat conditions which result from low blood and tissue levels of SAM-e. The author of this present invention fulfills these needs, and provides further related advantages.

### SUMMARY OF THE INVENTION

Briefly stated, the present invention discloses compositions of substantially optically pure enantiomeric forms of SAM-e, and methods for the use thereof. These new substantially optically pure enantiomeric forms of SAM-e of this present invention have utility in increasing blood and other tissue or fluid levels of SAM-e, as well as treating or preventing a wide variety of conditions associated with low blood or other tissue or fluid levels of SAM-e and inhibit tumor necrosis factor alpha. Thus in one embodiment, a substantially optically pure enantiomeric form of SAM-e salt is administered to a warm-blooded animal in need thereof to increase SAM-e levels. In another embodiment, a substantially optically pure enantiomeric form of SAM-e salt is administered to a warm-blooded animal in need thereof to prevent or treat a condition associated with low levels of SAM-e. In yet a further embodiment, a substantially optically pure enantiomeric form of SAM-e salt is administered to a warm blooded animal to prevent and or treat the following conditions: aging, aging of the skin, Alzheimer's disease, rheumatoid arthritis, osteoarthritis, both as an anti-inflammatory as well as to promote new cartilage formation, cancer, conditions of hypomethylation, mitochondrial diseases, hypomethylation of DNA and RNA, nerve damage associated with HIV/AIDS, anxiety, attention deficit disorder and ADHD, sleep regulation, organ preservation for transplant industry, dyslipidemias, excess sebum production, migraines, bile dysfunction caused by pregnancy and use of contraceptive medications, depression, acute and chronic liver disease, cirrhosis of the liver, ischemic reperfusion injury, Parkinson's disease, memory disturbances, memory loss, pancreatitis, intrahepatic cholestasis, inflammation, pain, side effects of administration of chemotherapy, liver disease associated with administration of total parenteral nutrition, liver dysfunction, low tissue levels of glutathione, administration of neuroleptic drugs, administration of cyclosporin A, asthma, alcohol withdrawal,

### DETAILED DESCRIPTION OF THE INVENTION

As mentioned above, this invention is generally directed to compositions of a substantially optically pure enantiomeric form of SAM-e salts. Such new optically pure enantiomeric forms of SAM-e salts, when administered to a warm blooded animal in need thereof, have utility in the prevention or treatment of conditions associated with low levels of SAM-e in warm blooded animals, including humans.

As used herein, the term “conditions” includes diseases, injuries, disorders, indications and/or afflictions that are associated with decreased levels of SAM-e. The term “treat” or “treatment” means that the symptoms associated with one or more conditions associated with low levels of SAM-e are alleviated or reduced in severity or frequency and the term “prevent” means that subsequent occurrences of such symptoms are avoided or that the frequency between such occurrences is prolonged.

The substantially optically pure enantiomeric forms of SAM-e salts of this invention may be used to prevent and/or treat a variety of conditions associated with lowered levels of SAM-e. Due to its ubiquitous distribution in mammalian tissue, SAM-e is associated with a variety of conditions: aging, aging of the skin, Alzheimer's disease, rheumatoid arthritis, osteoarthritis, both as an anti-inflammatory as well as to promote new cartilage formation, cancer, conditions of hypomethylation, mitochondrial diseases, hypomethylation of DNA and RNA, HIV/AIDS, anxiety, attention deficit disorder and ADHD, sleep regulation, organ preservation for transplant industry, dyslipidemias, excess sebum production, migraines, bile dysfunction caused by pregnancy and use of contraceptive medications, depression, acute and chronic liver disease, cirrhosis of the liver, ischemic reperfusion injury, Parkinson's disease, memory disturbances, memory loss, pancreatitis, intrahepatic cholestasis, inflammation, pain, side effects of administration of chemotherapy, liver disease associated with administration of total parenteral nutrition, liver dysfunction, low tissue levels of glutathione, administration of neuroleptic drugs, administration of cyclosporin A, asthma, alcohol withdrawal,.

Accordingly, substantially optically pure enantiomeric forms of SAM-e salts of this invention are effective in preventing and/or treating the above conditions due to their ability to increase SAM-e levels. To this end, substantially optically pure enantiomeric forms of SAM-e salts of the present invention may be used for pharmaceutical, prophylactic and/or cosmetic purposes, and are administered to a warm-blooded animal in an effective amount to achieve a desired result.

In the case of pharmaceutical administration, an effective amount is a quantity sufficient to treat the symptoms of a condition and/or the underlying condition itself An effective amount in the context of prophylactic administration means an amount sufficient to avoid or delay the onset of a condition and/or its symptoms. Lastly, an effective amount with regard to cosmetic administration is an amount sufficient to achieve the desired cosmetic result.

In a preferred embodiment, the substantially optically pure enantiomeric forms of SAM-e salts of the present invention are administered to a warm-blooded animal as a pharmaceutical, prophylactic or cosmetic composition containing at least one substantially optically pure enantiomeric forms of SAM-e salt in combination with at least one pharmaceutically, prophylactically or cosmetically acceptable carrier or diluent. Administration may be accomplished by systemic or topical application, with the preferred mode dependent upon the type and location of the conditions to be treated. Frequency of administration may vary, and is typically accomplished by daily administration.

Systemic administration may be achieved, for example, by injection (e.g., intramuscular, intravenous, subcutaneous or intradermal) or oral delivery of the composition to the warm-blooded animal. Suitable carriers and diluents for injection are known to those skilled in the art, and generally are in the form of an aqueous solution containing appropriate buffers and preservatives. Oral delivery is generally accomplished by formulating the composition in a liquid or solid form, such as a tablet or capsule, by known formulation techniques.

Topical administration may be accomplished, for example, by formulating the composition as solution, cream, gel, ointment, powder, paste, gum or lozenge using techniques known to those skilled in the formulation field. As used herein, topical administration includes delivery of the composition to mucosal tissue of the mouth, nose and throat by, for example, spray or mist application, as well as to the vagina and rectum by, for example, suppository application.

The following example illustrates the synthetic process by which the new enantiomeric SAM-e salts may be made. In addition, the example shows how these new SAM-e salts may be used clinically. This example is given to illustrate the present invention, but not by way of limitation. Accordingly, the scope of this invention should be determined not by the embodiment illustrated, but rather by the appended claims and their legal equivalents.

### EXAMPLE 1

1. (S, S)-s-adenosylmethionine was prepared according to the method of Hoffman (Hoffman, Chromatographic Analysis of the Chiral and Covalent Instability of S-adenosyl-l-methionine, Biochemistry 1986, 25 4444-4449). Enantiomerically pure (S, S)-SAM-e was stabilized according to Fiecchi (U.S. Pat. No. 4,028,183, Jun. 7, 1977) using p-toluene sulfonate as the stabilizing agent.

(S, S)-s-adenosylmethionine 400 mg was administered twice daily in an open, non-blind study of 10 volunteers who gave informed consent. All patients had normal results on pre-study medical examinations, including laboratory examinations. Patients received 400 mg of (S, S)-s-adenosylmethionine in an enteric-coated tablet form twice daily for 14 days or until remission of depression symptoms. The 10 patients satisfied the DSM-III criteria for a major depressive episode. Patients' symptoms were monitored daily using the Hamilton Rating Scale for Depression. 9 patients completed the study. (One patient declined to continue the study after beginning.) Eight of the nine patients who completed the trial improved over the 14 days. One patient had no change at all. No side effects were noted or reported by any of the patients nor as measured by laboratory or physical examination. (S, S)-s-adenosylmethionine 400 mg twice daily appeared to be safe and effective in this small, non-blinded study of depression.

I claim:

1. A pharmaceutical composition comprising substantially optically pure enantiomer (S,S) s-adenosylmethionine or a defined non-racemic ratio of (S, S)-s-adenosylmethionine: (R,S)-s-adenosylmethionine, their pharmaceutically acceptable salts and a pharmaceutically acceptable carrier

2. A pharmaceutical composition as described in claim 1 wherein the defined non-racemic ratio of (S,S)-s-adenosylmethionine: (R,S)-s-adenosylmethionine is about 80% to about 100%: about 20% to about 0% by weight respectively.

3. A pharmaceutical composition as described in claim 1 wherein the defined non-racemic ratio of (S,S) s-adenosylmethionine: (R,S)-s-adenosylmethionine is about 95% to about 100%: about 5% to about 0% by weight respectively.

4. A pharmaceutical composition as described in claim 1 wherein the pharmaceutically acceptable salt for each enantiomer is selected from the group consisting of: a lipophilic salt of S-adenosyl-L-methionine(SAM) of the formula SAM.sup.n+[R—CO—NH—(CH.sub.2).sub.2—SO.sup.-.sub.3].sub.n in which R—CO is a member selected from the group consisting of C.sub.12-C.sub.26 saturated and unsaturated, linear and branched acyl and C.sub.12-C.sub.26 cycloalkyl-substituted acyl, and n is an integer from 3 to 6 according to the SAM charge; double salts corresponding to the formula SAM.sup.+.HSO.sub.4.sup.-.H.sub.2 SO.sub.4 .2 CH.sub.3 C.sub.6 H.sub.4 SO.sub.3 H.; salts (S,S)-s-adenosylmethionine with sulphonic acids selected from the group consisting of methanesulphonic, ethanesulphonic, 1-n-dodecanesulphonic, 1-n-octadecanesulphonic, 2-chloroethanesulphonic, 2-bromoethanesulphonic, 2-hydroxyethanesulphonic, 3-hydroxypropanesulphonic, d-,1-,d,1-10-camphorsulphonic, d-,1-,d,1-3-bromocamphor-10-sulphonic, cysteic, benzenesulphonic, p-chlorobenzenesulphonic, 2-mesitylbenzenesulphonic, 4-biphenylsulphonic, 1-naphthalenesulphonic, 2-naphthalenesulphonic, 5-sulphosalicylic, p-acetylbenzenesulphonic, 1,2-ethanedisulphonic, methanesulphonic acid, ethanesulphonic acid, 1-n-dodecanesulphonic acid, 1-n-octadecanesulphonic acid, 2-chloroethanesulphonic acid, 2-bromoethanesulphonic acid, 2-hydroxyethanesulphonic acid, d-,1-,d,1-10-camphorsulphonic acid, d-,1-,d,1-3-bromocamphor-10-sulphonic acid, cysteic acid, benzenesulphonic acid, 3-hydroxypropanesulphonic acid, 2-mesitylbenzenesulphonic acid, p-chlorobenzenesulphonic acid, 4-biphenylsulphonic acid, 2-naphthalenesulphonic acid, 5-sulphosalicylic acid, 1,2-ethanedisulphonic acid, p-acetylbenzenesulphonic acid, 1-naphthalenesulphonic acid, o-benzenedisulphonic and chondroitinesulphuric acids, and double salts of said acids with sulphuric acid; S-adenosyl-L-methionine or a pharmaceutically acceptable salt thereof and an effective amount of a lithium salt selected from the group consisting of lithium chloride, lithium bromide, lithium iodide, lithium sulfate, lithium nitrate, lithium phosphate, lithium borate, lithium carbonate, lithium formate, lithium acetate, lithium citrate, lithium succinate and lithium benzoate; water-soluble salt of a bivalent or trivalent metal is a member selected from the group consisting of calcium chloride, ferric chloride, magnesium chloride, and magnesium sulfate; the salt of S-adenosyl-L-methionine is a member selected from the group consisting of salts of S-adenosyl-L-methionine with hydrochloric acid, sulfuric acid, p-toluenesulfonic acid, phosphoric acid, formic acid, acetic acid, citric acid, tartaric acid, and maleic acid; and a double salt of S-adenosyl-L-methionine with said acids; a salt of S-adenosyl-L-methionine and a water-soluble polyanionic substance selected from the group consisting of a polyphosphate, metaphosphate, polystyrene sulfonate, polyvinyl sulfonate, polyvinyl sulfate, polyvinyl phosphate, and polyacrylate wherein the stoichiometric ratio of mols of S-adenosyl-L-methionine to gram-equivalent of the polyanionic substance is from 0.1:1 to 0.5; a salt of S-adenosyl-L-methionine wherein the polyanionic substance is a polyphosphate, para-polystyrene sulfonate or metaphosphate; a salt of the general formula: SAM-e.nR(O).sub.m (SO.sub.3 H)p (I) where m can be zero or 1; n is 1.5 when p is 2, and is 3 when p is 1; R is chosen from the group consisting of alkyl, phenylalkyl and carboxyalkyl, in which the linear or branched alkyl chain contains from 8 to 18 carbon atoms, and in particular for producing SAM-e salts of sulphonic acids, or of sulphuric acid esters, or of dioctylsulphosuccinic acid;

5. A pharmaceutical composition as described in claim 1 wherein the pharmaceutically acceptable salt for each enantiomer is selected from the group consisting of bisulfate; tri-p-toluenesulfonate; chloride, carbonate, bicarbonate, bromide, chloride, iodide, hydrochloride.

6. A pharmaceutical composition as described in claim 1 wherein the pharmaceutically acceptable salt for each enantiomer is selected from the group consisting of double and single salts of S-adenosyl-L-methionine with sulphuric acid and p-toluenesulphonic acid.

7. A pharmaceutical composition as described in claim 1 wherein the pharmaceutically acceptable salt for each enantiomer is 1,4 butane disulfonate.

8. A method of claim 1 wherein the composition of claim 1 is administered to a warm-blooded animal to treat a condition of lowered s-adenosylmethionine levels by increasing s-adenosylmethionine levels, comprising administering to an animal in need thereof an effective amount of the composition of claim 1.

9. The method of claim 8 wherein the condition to be treated is selected from the group consisting of: ageing, ageing of the skin, Alzheimer's disease, osteoarthritis, rheumatoid arthritis, cancer, conditions of hypomethylation, mitochondrial diseases, hypomethylation of DNA and RNA, HIV/AIDS, anxiety, attention deficit disorder and ADHD, sleep dysregulation, organ preservation, dyslipidemias, excess sebum production, migraines, bile dysfunction, bile dysfunction caused by pregnancy and use of contraceptive medications, depression, acute and chronic liver disease, alcohol liver disease, hepatitis B and C, cirrhosis of the liver, ischemic reperfusion injury, strokes, Parkinson's disease, MS, memory disturbances, impaired memory, memory loss, pancreatitis, intrahepatic cholestasis, inflammation, pain, side effects of administration of chemotherapy, total parenteral nutrition induced liver disease, increased levels of tumor necrosis factor alpha, seborrhea, dermatitis, peripheral occlusive arterial disease, low glutathione levels, administration of neuroleptic drugs, administration of cyclosporin A, asthma, alcohol withdrawal.

10. The method of claim 8 wherein the condition to be prevented is selected from the group consisting of: ageing, ageing of the skin, Alzheimer's disease, osteoarthritis, rheumatoid arthritis, cancer, conditions of hypomethylation, mitochondrial diseases, hypomethylation of DNA and RNA, HIV/AIDS, anxiety, attention deficit disorder and ADHD, sleep dysregulation, organ preservation, dyslipidemias, excess sebum production, migraines, bile dysfunction, bile dysfunction caused by pregnancy and use of contraceptive medications, depression, acute and chronic liver disease, alcohol liver disease, hepatitis B and C, cirrhosis of the liver, ischemic reperfusion injury, strokes, Parkinson's disease, MS, memory disturbances, impaired memory, memory loss, pancreatitis, intrahepatic cholestasis, inflammation, pain, side effects of administration of chemotherapy, total parenteral nutrition induced liver disease, increased levels of tumor necrosis factor alpha, seborrhea, dermatitis, peripheral occlusive arterial disease, low glutathione levels, administration of neuroleptic drugs, administration of cyclosporin A, asthma, alcohol withdrawal.

11. The method of claim 8 wherein the route of administration of the composition is chosen from the group consisting of topically, systemically, orally, intranasally, rectally, transdermally,

12. The method of claim 8 wherein the composition can be administered together with another drug selected from the group consisting of levodopa, cyclosporin A, ibuprofen, aspirin, methotrexate, a neuroleptic, vitamin B, folic acid

13. A method of claim 1 wherein the composition of claim 1 is administered to a warm-blooded animal to treat a condition of lowered anti-oxidant levels by increasing said antioxidant levels comprising administering to an animal in need thereof an effective amount of the composition of claim 1.
